SEQUENCE LISTING (2 sequences)

SEQ ID NO: 1 - protein, 908 aa, Mus musculus
MPSSGPGDTSSSSLEREDDRKEGEEQEENRGKEERQEPSATARKVGRPGRKRKHPPVESSDTPKDPAVTTKSQPMAQDSGPSDLLPNGDLEKRSEPQPEEGSPAAGQKGGAPAEGEGTETPPEASRAVENGCCVTKEGRGASAGEGKEQKQTNIESMKMEGSRGRLRGGLGWESSLRQRPMPRLTFQAGDPYYISKRKRDEWLARWKREAEKKAKVIAVMNAVEENQASGESQKVEEASPPAVQQPTDPASPTVATTPEPVGGDAGDKNATKAADDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPEEEKNPYKEVYTDMWVEPEAAAYAPPPPAKKPRKSTTEKPKVKEIIDERTRERLVYEVRQKCRNIEDICISCGSLNVTLEHPLFIGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 2 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

(19) United States
(12) Patent Application Publication
(10) Pub. No.: US 2026/0060991 A1
(43) Pub. Date: Mar. 5, 2026

(54) METHODS AND COMPOSITIONS FOR INHIBITING CLONAL HEMATOPOIESIS

(71) Applicant: The Jackson Laboratory, Bar Harbor, ME (US)

(72) Inventor: Jennifer Trowbridge, Bar Harbor, ME (US)

(73) Assignee: The Jackson Laboratory, Bar Harbor, ME (US)

(21) Appl. No.: 19/107,183

(22) PCT Filed: Aug. 28, 2023
(86) PCT No.: PCT/US2023/072977

§ 371 Date: Feb. 27, 2025

Related U.S. Application Data
(60) Provisional application No. 63/402,828, filed on Aug. 31, 2022.

(51) Int. Cl. A61K 31/635; A61K 31/353; A61K 31/506; A61P 35/02

(52) U.S. Cl. CPC A61K 31/635; A61K 31/353; A61K 31/506; A61P 35/02

(57) ABSTRACT

Clonal hematopoiesis is an age-related condition caused by somatic mutations that give a hematopoietic stem cell a clonal selective advantage. While clonal hematopoiesis is a benign condition, individuals affected by it have an increased risk of developing blood cancers, such as acute myeloid leukemia (AML). The present disclosure provides, in some aspects, methods for inhibiting clonal hematopoiesis using a senolytic agent to target senescence of bone marrow stromal cells.

### RELATED APPLICATION

This application claims the benefit under 35 U.S.C. § 119 (e) of U.S. provisional application No. 63/402,828, filed on Aug. 31, 2022, which is incorporated by reference herein in its entirety.

### GOVERNMENT LICENSE RIGHTS

This invention was made with government support under DK118072, AG069010, and AG077925 awarded by National Institutes of Health. The government has certain rights in the invention.

### REFERENCE TO AN ELECTRONIC SEQUENCE LISTING

The contents of the electronic sequence listing (J022770120WO00-SEQ-VLJ.xml; Size: 4,597 bytes; and Date of Creation: Aug. 10, 2023) is herein incorporated by reference in its entirety.

### BACKGROUND

Clonal hematopoiesis is an age-related condition caused by somatic mutations that give a hematopoietic stem cell a clonal selective advantage. While clonal hematopoiesis is a benign condition, individuals affected by it have an increased risk of developing blood cancers, such as acute myeloid leukemia (AML). The most frequently mutated gene in human clonal hematopoiesis and AML is DNA methyltransferase 3A (DNMT3A). The precise mechanisms by which DNMT3A mutant gene confers a clonal advantage and leads to development of AML is unknown.

### SUMMARY

The present disclosure provides, in some aspects, methods for inhibiting clonal hematopoiesis using a senolytic agent to target senescence of bone marrow stromal cells. The studies described herein demonstrate that subsets of bone marrow stromal cells gain an enriched gene signature of cellular senescence and exhibit increased expression of senescence biomarkers in the context of Dnmt3a-mutant hematopoiesis. Without being bound by theory, the data provided herein suggests that Dnmt3a-mutant hematopoietic stem and progenitor cells (HSPCs) induce senescence of the bone marrow microenvironment to favor clonal expansion and transformation to AML. Ex vivo cocultures showed that bone marrow stromal cells cultured with Dnmt3a-mutant HSPCs have increased expression of senescence markers and senescence associated secretory phenotype compared to bone marrow stromal cells cultured with control wildtype HSPCs. Functional studies performed to assess the importance of bone marrow stromal cell senescence in Dnmt3a-mutant transformation to AML showed that treatment with a senolytic agent reduced peripheral blood myeloid cell overproduction concurrent with transformation of Dnmt3a-mutant cells, suggesting that treatment with senolytic agents delays AML development. Thus, the data provided herein shows that Dnmt3a-mutant hematopoietic cells can induce bone marrow stromal cell senescence, and that targeting the senescent bone marrow microenvironment using a senolytic agent, for example, is a viable strategy for inhibiting clonal hematopoiesis and preventing the development of (e.g., transformation to) AML.

Some aspects provide a method of inhibiting clonal hematopoiesis in a subject in need thereof, the method comprising administering a senolytic agent to subject in an effective amount to inhibit senescence of bone marrow stromal cells in the subject, thereby inhibiting clonal hematopoiesis in the subject, relative to a control.

In some embodiments, the subject exhibits one or more symptom of acute myeloid leukemia.

In some embodiments, the subject has one or more known risk factors associated with acute myeloid leukemia.

In some embodiments, the subject has acute myeloid leukemia.

In some embodiments, the subject is 50 years old or older.

In some embodiments, the effective amount of the senolytic agent inhibits myeloproliferation of the bone marrow stromal cells.

In some embodiments, the effective amount of the senolytic agent inhibits progression from clonal hematopoiesis to myeloid malignancy, relative to a control.

In some embodiments, hematopoietic stem and progenitor cells (HSPCs) of the subject comprise somatic mutation that gives the HSPCs a clonal selective advantage.

In some embodiments, the mutation is a DNA methyltransferase 3A (DNMT3A) mutation.

In some embodiments, the method further comprises assaying a sample from the subject for presence of the somatic mutation.

In some embodiments, the method further comprises assaying a sample from the subject increased expression of senescence markers, relative to a control.

In some embodiments, the senescence markers are selected from senescence-associated β-galactosidase (SA-β-Gal), Cdkn2a (P16), Cdkn1a (P21), Cdkn1b (P27), IL-6 and IL-1a.

In some embodiments, the bone marrow stromal cells comprise adipo-Cxc112-abundant reticular (CAR) cells and osteo-CAR cells.

In some embodiments, the senolytic agent is selected from dasatinib, quercetin, fisetin, 17-DMAG, navitoclax, and catechins.

In some embodiments, the senolytic agent is administered via an intravenous route or an intraosseous route.

Some aspects relate to a method of inhibiting clonal hematopoiesis in a subject in need thereof, the method comprising: assaying a biological sample obtained from the subject for a DNA methyltransferase 3A (DNMT3A) mutation; and administering a senolytic agent, optionally selected from dasatinib, quercetin, fisetin, 17-DMAG, navitoclax, and catechins, to subject in an effective amount to inhibit senescence of bone marrow stromal cells in the subject, thereby inhibiting clonal hematopoiesis in the subject, relative to a control.

Some aspects relate to a method of inhibiting clonal hematopoiesis in a subject in need thereof, the method comprising: assaying a biological sample obtained from the subject for a biomarker of cell senescence; and administering a senolytic agent, optionally selected from dasatinib, quercetin, fisetin, 17-DMAG, navitoclax, and catechins, to subject in an effective amount to inhibit senescence of bone marrow stromal cells in the subject, thereby inhibiting clonal hematopoiesis in the subject, relative to a control.

In some embodiments, the subject is at risk for developing a myeloid malignancy. A subject may be at risk for developing a myeloid malignancy if, for example, the subject has a family history of myeloid malignancy.

In some embodiments, the biological sample comprises hematopoietic cells, optionally bone marrow stromal cells.

In some embodiments, the assaying comprises performing single-cell RNA sequencing (RNA-seq) on the biological sample.

### BRIEF DESCRIPTION OF THE DRAWINGS

FIG. 1A-1B: Cellular senescence gene signature is enriched in the bone marrow stromal cells (BMSCs) of Dnmt3a-mutant (Dnmt3a^{R8781/+}) mice. (FIG. 1A) Fold change of P21 gene expression in the Adipo-CAR and Osteo-CAR BMSC population at 12 weeks post-transplant of control wild type or Dnmt3a-mutant hematopoietic cells into wild type recipient animals. (FIG. 1B) Enrichment of cellular senescence pathway in the Adipo-CAR and Osteo-CAR BMSC population at 12 weeks post-transplant of control (WT) or Dnmt3a-mutant hematopoietic cells into wild type recipient animals.

FIG. 2A-2B: BMSCs cultured with Dnmt3a-mutant hematopoietic stem and progenitor cells (HSPCs) induce increased expression of senescence. Gene expression of senescence markers (P16, P21) (FIG. 2A) and beta-galactosidase (β-gal) SASP (IL-6, IL-1a) (FIG. 2B) in BMSCs cultured control HSPCs or Dnmt3a-mutant HSPC for 7 days.

FIG. 3A-3B: Dnmt3a-mutant HSPCs induce senescence in BMSCs. Senescence-associated beta-galactosidase (β-gal) expression in the BMSCs (FIG. 3A) and endothelial cells (FIG. 3B) from MxCre control or Dnmt3a-mutant hematopoietic cells transplanted into wild type recipient animals 12 weeks post-transplant.

FIG. 4A-4B: Dnmt3a-mutant HSPCs cause increase expression of the anti-apoptotic proteins BCL2 and BCLxL in BMSCs. BCL2 and BCLxL protein expression in the BMSCs from MxCre control or Dnmt3a-mutant hematopoietic cells transplanted into wild type recipient animals 12 weeks post-transplant (FIG. 4A). Number of BMSCs at 12 weeks post-transplant of MxCre control (WT) or Dnmt3a-mutant hematopoietic cells into wild type recipient animals (FIG. 4B).

FIG. 5: Dnmt3a-mutant expand hematopoictic stem cell (HSC) pool. Frequency of donor-derived HSCs at 12 weeks post-transplant of MxCre control (WT) or Dnmt3a-mutant hematopoietic cells into wild-type recipient animals.

FIG. 6A-6E: Navitoclax treatment attenuates Dnmt3a-mutant peripheral blood myeloid cell overproduction. (FIG. 6A) Dnmt3a-mutant cells were transplanted into wild type recipient mice, 12 weeks post-transplant Navitoclax or vehicle control was administered, and 2 weeks later the Npm1 mutation was induced in the Dnmt3a-mutant cells. (FIG. 6B) Frequency of donor-derived mature Dnmt3a;Npm1-mutant myeloid cells in peripheral blood of Navitoclax or vehicle control treated transplant mice. (FIG. 6C) Engraftment of donor-derived mature Dnmt3a;Npm1-mutant myeloid cells in BM (FIG. 6D) β-gal expression in BMSCs, and (FIG. 6E) monocyte count in navitoclax versus vehicle treated mice.

### DETAILED DESCRIPTION

### I. Clonal Hematopoiesis

Provided herein, in some aspects, are methods and compositions for inhibiting (e.g., preventing or suppressing) clonal hematopoiesis in a subject in need thereof by administering a (one or more) senolytic agent in an effective amount to inhibit senescence of bone marrow stromal cells (BMSCs) in the subject, thereby inhibiting clonal hematopoiesis in the subject. BMSCs reside in the bone marrow microenvironment, which is the collection of cells and structures that together support blood cell production in the bone marrow. The bone marrow has two cell fractions, hematopoietic and non-hematopoietic cell fractions. The hematopoietic cell fraction expresses CD45 (CD45+). The non-hematopoietic cell fraction lacks the expression of the hematopoictic and erythrocyte markers CD45 and Ter119 (CD45− Ter119−). This microenvironment also includes hematopoietic stem and progenitor cells (HSPCs) (including hematopoietic stem cells (HSCs)).

Bone marrow stromal cells (or stroma) include a heterogeneous population of cells that provide the structural and physiological support for HSPCs. The BMSCs also contain cells with a stem-cell-like character that allows them to differentiate into bone, cartilage, adipocytes, and hematopoictic supporting tissues. Additional cell types that reside in the bone marrow stroma include, but are not limited to, fibroblasts, macrophages, adipocytes, osteoblasts, osteoclasts and endothelial cells. Bone marrow stromal cells also include adipo-Cxcl12-abundant reticular (CAR) cells and osteo-CAR cells, endothelial cells (EC), EC-Arteriolar, and EC-Sinusoidal. In some embodiments, the bone marrow stromal cells are adipo-Cxcl12-abundant reticular (CAR) cells and/or osteo-CAR cells.

Hematopoietic stem cells are capable of self-renewal and have the capacity to reconstitute all types of blood cells, including white blood cells, red blood cells, and platelets and respond to changing needs for blood cells in peripheral tissues. Hematopoictic stem and progenitor cells, while primarily residing in the bone marrow, can traffic to other hematopoietic and nonhematopoietic organs. When somatic mutations accumulate in HSCs and undergo positive selection (e.g., clonal selective advantage) this leads to clonal HSC expansion called clonal hematopoiesis. As a result, the blood cells generated by clonal hematopoiesis will all have the same genetic mutation and a different genetic pattern than the pre-existing blood cells. Most people with clonal hematopoiesis do not show symptoms of disease. However, people with clonal hematopoiesis have an increased risk of developing cardiovascular disease and blood cancers, such as myelodysplastic syndrome and acute myeloid leukemia. No single cause of clonal hematopoiesis has been identified, but characteristics that can increase risk of developing clonal hematopoiesis include age, smoking and being male and white. Additionally, radiation therapy and some chemotherapies may be linked to clonal hematopoiesis. Aging is a main driving factor of clonal hematopoiesis, the percentage of people under the age of 50 years that have clonal hematopoietic somatic mutations is only 1%, however individuals older than 65 years are at a 10% risk, which jumps to about 20% of those older than 90 years old. The most frequently mutated genes include DNA methyltransferase 3A (“DNMT3A”), Tet Methylcytosine Dioxygenase 2 (“TET2”), Janus Kinase 2 (“JAK2”), and ASXL Transcriptional Regulator 1 (“ASXL1”).

Thus, “inhibiting clonal hematopoiesis” refers to the process of preventing or delaying clonal HSC expansion. As discussed herein, certain mutations in HSPC induce senescence to favor clonal expansion and subsequent transformation to acute myeloid leukemia. Using a senolytic agent to prevent this senescence in turn prevents clonal expansion.

In some embodiments, HSCs of a subject comprise a somatic mutation that gives the HSCs a clonal selective advantage. A clonal selective advantage occurs when a somatic variant has better fitness compared to a non-variant and is positively selected for. A somatic variant is a cell that has a somatic mutation. Somatic mutations are alterations of DNA that occurs in a somatic cell of a multicellular organism. A somatic cell is any cell that is not a germline cell (e.g., gamete, germ cell or gametocyte). Somatic mutations do not pass to offspring because they do not occur in germline cells. Somatic mutations may occur throughout the life cycle of an organism either spontaneously, in response to stress, or accumulate because of errors in DNA repair. In some embodiments, a somatic mutation is in a HSPC and gives the HSPC a clonal selective advantage. In some embodiments, the somatic mutation is a mutation of the DNMT3A gene. In some embodiments, the somatic mutation is a mutation of the mouse DNMT3A gene. In some embodiments, the somatic mutation is a mutation of the human DNMT3A gene. In humans, most mutations in DNMT3A are heterozygous point mutations within the catalytic domain at DNMT3A residue R882 (equivalent to R878 in mouse), which is proposed to induce a dominant-negative loss of de novo methylation activity. In some embodiments, the somatic mutation is homologous to (e.g., orthologous to) a somatic mutation at amino acid position 878 relative to a wild-type mouse DNMT3A (UniProt Accession No. 088508; SEQ ID NO: 1). In some embodiments, the mutation is homologous to (e.g., orthologous to) an R878H mutation in the mouse DNMT3A protein. In some embodiments, the somatic mutation is at amino acid position 882 relative to a wild-type human DNMT3A (UniProt Accession No. Q9Y6K1; SEQ ID NO: 2). In some embodiments, the somatic mutation is a R882H mutation in the human DNMT3A protein.

### Amino Acid Sequence of Wild-Type Mouse DNMT3A

(SEQ ID NO: 1)
MPSSGPGDTSSSSLEREDDRKEGEEQEENRGKEERQEPSATARKVGRPG
RKRKHPPVESSDTPKDPAVTTKSQPMAQDSGPSDLLPNGDLEKRSEPQP
EEGSPAAGQKGGAPAEGEGTETPPEASRAVENGCCVTKEGRGASAGEGK
EQKQTNIESMKMEGSRGRLRGGLGWESSLRQRPMPRLTFQAGDPYYISK
RKRDEWLARWKREAEKKAKVIAVMNAVEENQASGESQKVEEASPPAVQQ
PTDPASPTVATTPEPVGGDAGDKNATKAADDEPEYEDGRGFGIGELVWG
KLRGFSWWPGRIVSWWMTGRSRAAEGTRWVMWFGDGKFSVVCVEKLMPL
SSFCSAFHQATYNKQPMYRKAIYEVLQVASSRAGKLFPACHDSDESDSG
KAVEVONKQMIEWALGGFQPSGPKGLEPPEEEKNPYKEVYTDMWVEPEA
AAYAPPPPAKKPRKSTTEKPKVKEIIDERTRERLVYEVRQKCRNIEDIC
ISCGSLNVTLEHPLFIGGMCQNCKNCFLECAYQYDDDGYQSYCTICCGG
REVLMCGNNNCCRCFCVECVDLLVGPGAAQAAIKEDPWNCYMCGHKGTY
GLLRRREDWPSRLQMFFANNHDQEFDPPKVYPPVPAEKRKPIRVLSLFD
GIATGLLVLKDLGIQVDRYIASEVCEDSITVGMVRHQGKIMYVGDVRSV
TQKHIQEWGPFDLVIGGSPCNDLSIVNPARKGLYEGTGRLFFEFYRLLH
DARPKEGDDRPFFWLFENVVAMGVSDKRDISRFLESNPVMIDAKEVSAA
HRARYFWGNLPGMNRPLASTVNDKLELQECLEHGRIAKFSKVRTITTRS
NSIKQGKDQHFPVFMNEKEDILWCTEMERVFGFPVHYTDVSNMSRLARQ
RLLGRSWSVPVIRHLFAPLKEYFACV

### Amino Acid Sequence of Wild-Type Human DNMT3A

(SEQ ID NO: 2)
MPAMPSSGPGDTSSSAAEREEDRKDGEEQEEPRGKEERQEPSTTARKVG
RPGRKRKHPPVESGDTPKDPAVISKSPSMAQDSGASELLPNGDLEKRSE
PQPEEGSPAGGQKGGAPAEGEGAAETLPEASRAVENGCCTPKEGRGAPA
EAGKEQKETNIESMKMEGSRGRLRGGLGWESSLRQRPMPRLTFQAGDPY
YISKRKRDEWLARWKREAEKKAKVIAGMNAVEENQGPGESQKVEEASPP
AVQQPTDPASPTVATTPEPVGSDAGDKNATKAGDDEPEYEDGRGFGIGE
LVWGKLRGFSWWPGRIVSWWMTGRSRAAEGTRWVMWFGDGKFSVVCVEK
LMPLSSFCSAFHQATYNKQPMYRKAIYEVLQVASSRAGKLFPVCHDSDE
SDTAKAVEVONKPMIEWALGGFQPSGPKGLEPPEEEKNPYKEVYTDMWV
EPEAAAYAPPPPAKKPRKSTAEKPKVKEIIDERTRERLVYEVRQKCRNI
EDICISCGSLNVTLEHPLFVGGMCQNCKNCFLECAYQYDDDGYQSYCTI
CCGGREVLMCGNNNCCRCFCVECVDLLVGPGAAQAAIKEDPWNCYMCGH
KGTYGLLRRREDWPSRLQMFFANNHDQEFDPPKVYPPVPAEKRKPIRVL
SLFDGIATGLLVLKDLGIQVDRYIASEVCEDSITVGMVRHQGKIMYVGD
VRSVTQKHIQEWGPFDLVIGGSPCNDLSIVNPARKGLYEGTGRLFFEFY
RLLHDARPKEGDDRPFFWLFENVVAMGVSDKRDISRFLESNPVMIDAKE
VSAAHRARYFWGNLPGMNRPLASTVNDKLELQECLEHGRIAKFSKVRTI
TTRSNSIKQGKDQHFPVFMNEKEDILWCTEMERVFGFPVHYTDVSNMSR
LARQRLLGRSWSVPVIRHLFAPLKEYFACV

### II. Myeloid Malignancies (e.g., Blood Cancer)

Aspects of the present disclosure provide methods that inhibit the progression from clonal hematopoiesis to a myeloid malignancy, such as blood cancer. Myeloid malignancies are a group of disorders characterized by the abnormal growth and differentiation of myeloid progenitor cells in the bone marrow and blood. The myeloid lineage in the hematopoietic system gives rise to various types of blood cells including red blood cells, some types of white blood cells (like neutrophils, eosinophils, and monocytes), and platelets. Acute Myeloid Leukemia (AML) is a rapidly progressing disease in which the bone marrow produces abnormal myeloblasts (a type of immature white blood cell), red blood cells, or platelets. It is the most common type of acute leukemia in adults. Chronic Myeloid Leukemia (CML) is characterized by the increased and unregulated growth of myeloid cells in the bone marrow and their accumulation in the blood. It has a specific genetic marker known as the Philadelphia chromosome, which is a result of a chromosomal translocation. Myelodysplastic Syndromes (MDS) are a diverse collection of conditions in which immature blood cells in the bone marrow do not mature or become healthy blood cells. This can lead to a low number of one or more types of blood cells. MDS can progress to AML in some cases. Myeloproliferative Neoplasms (MPN) are diseases in which the bone marrow produces too many red blood cells, white blood cells, or platelets. Examples include polycythemia vera (PV), essential thrombocythemia (ET), and primary myelofibrosis (PMF). Chronic Myelomonocytic Leukemia (CMML) is a type of leukemia that starts in blood-forming cells of the bone marrow and invades the blood. It affects monocytes and can spread to other parts of the body.

Blood cancers, also referred to as hematologic cancers, start in the bone marrow, which is where blood cells are produced. Normally functioning blood cells fight off infections and produce new blood cells. Blood cancers occur when abnormal blood cells grow (e.g., proliferate) out of control and interrupt the function of normal blood cells. There are three main types of blood cancer, leukemia, lymphoma and myeloma. Leukemia originates in the blood and bone marrow and occurs when the body creates too many abnormal white blood cells. When leukemia occurs, the bone marrow's ability to produce red blood cells and platelets are diminished. Non-Hodgkin and Hodgkin lymphomas are blood cancers that develop in the lymphatic system from white blood cells called lymphocytes. Hodgkin lymphoma is characterized by the presence of an abnormal lymphocyte called the Reed-Sternberg cell. Myeloma is a blood cancer of the blood's plasma cells which, is a type of white blood cell made in the bone marrow. Symptoms of these blood cancers include fever and frequent infections, fatigue, nausea, unexplained weight loss, bone/joint pain, headaches, shortness of breath and swollen lymph nodes. Blood cancers account for approximately 10% of cancer diagnoses with over 900,000 people worldwide diagnosed with blood cancer every year. In the United States, 68,000 people die from blood cancers every year. People with clonal hematopoiesis have an increased risk of developing blood cancers, specifically myelodysplastic syndrome (also called preleukemia) and acute myeloid leukemia. The transformation from clonal hematopoiesis to AML, for example, occurs through a process referred to as myeloproliferation. Myeloproliferation is the uncontrolled overproduction of one or more types of bone marrow cells. In some embodiments, the methods provided herein are used to inhibit (e.g., prevent or delay) the myeloproliferation that occurs during the transformation from clonal hematopoiesis to AML. In some embodiments, a senolytic agent, as described elsewhere herein, is used in an effective amount to inhibit myeloproliferation, for example, myeloproliferation of bone marrow stromal cells.

In some embodiments, a subject described herein has acute myeloid leukemia (AML). AML is a form of blood cancer that is characterized by infiltration of the bone marrow, blood and other tissues by proliferative, clonal, and abnormally or poorly differentiated cells of the hematopoietic system. The major categories of AML include, but are not limited to, AML with recurrent genetic abnormalities, AML with myelodysplasia-related changes, therapy-related AML, and AML not otherwise specified. AML accounts for about 1% of all cancers and is the most common form of acute leukemia among adults. An estimated 20,050 people are diagnosed with AML each year in the United States. Each year, approximately 11,540 deaths occur in the United States due to AML. The median age of diagnosis is 67 years, with 54% of patients diagnosed at 65 years or older. In some embodiments, a subject herein is 50 years old or older (e.g., 55, 60, 65, 70, 75, or 80 years old). In some embodiments, a subject herein is 50 years old or older, 55 years old or older, 60 years old or older, 65 years old or older, 70 years old or older, 75 years old or older. In some embodiments, a subject herein is 50 years old or older. In some embodiments, a subject herein is 55 years old or older. In some embodiments, a subject herein is 60 years old or older. In some embodiments, a subject herein is 65 years old or older. In some embodiments, a subject herein is 70 years old or older. In some embodiments, a subject herein is 75 years old or older. In some embodiments, a subject herein is 80 years old or older. The general therapeutic strategy to treat patients with AML has not changed substantially in over 30 years. Current therapies include intensive induction therapy, consolidation with intensive chemotherapy and allogenic hematopoietic cell transplantation. For older patients that cannot receive intensive chemotherapy only supportive care is available and the median survival is 5 to 6 months. Therefore, additional therapies for AML are needed.

In some embodiments, a subject exhibits one or more symptom(s) of AML. Symptoms of AML progress over several weeks. Symptoms of AML include, but are not limited to, fatigue, breathlessness, fever, pale skin, sweating, losing weight, frequent infections, unusual and frequent bleeding, bruising, bone and joint pain and swollen glands. In some embodiments, a subject exhibit at least 1, at least 2, at least 3, at least 4, at least 5, at least 6, at least 7, at least 8, at least 9, or at least 10 symptoms of AML. In some embodiments, a subject exhibit at least 1 symptom of AML. In some embodiments, a subject exhibit at least 2 symptoms of AML. In some embodiments, a subject exhibit at least 3 symptoms of AML. In some embodiments, a subject exhibit at least 4 symptoms of AML. In some embodiments, a subject exhibit at least 5 symptoms of AML. In some embodiments, a subject exhibit at least 6 symptoms of AML. In some embodiments, a subject exhibit at least 7 symptoms of AML. In some embodiments, a subject exhibit at least 8 symptoms of AML. In some embodiments, a subject exhibit at least 9 symptoms of AML. In some embodiments, a subject exhibit at least 10 symptoms of AML.

In some embodiments, a subject has one or more risk factor(s) associated with acute myeloid leukemia. A risk factor is a factor that influences the likelihood of getting a disease. Having one or several risk factors may not mean that a subject will develop AML. Conversely, a subject may develop AML without having a risk factor. Known risk factors of AML include but are not limited to aging (old age is a risk factor), being male, smoking, exposure to certain chemicals (e.g., benzene, formaldehyde, diesel, or gasoline), treatment with chemotherapy drugs, exposure to radiation, having a blood disorder (e.g., polycythemia vera, essential thrombocythemia, idiopathic myelofibrosis, or myelodysplastic syndrome), family history of the disease, exposure to electromagnetic fields, and exposure to herbicides or pesticides. Additionally, there are several genetic syndromes that are risk factors for AML, for example, Fanconi anemia, Bloom syndrome, Ataxia-telangiectasia, Diamond-Blackfan anemia, Shwachman-Diamond syndrome, Li-Fraumeni syndrome, Neurofibromatosis type 1, Severe congenital neutropenia. In some embodiments, a subject has one or more (e.g., one or more, two or more, three or more, four or more, five or more, six or more, seven or more, eight or more, nine or more, ten or more) risk factor(s) associated with acute myeloid leukemia. In some embodiments, a subject has one or more, two or more, three or more, four or more, five or more, six or more, seven or more, eight or more, nine or more, ten or more risk factor(s) associated with acute myeloid leukemia. In some embodiments, a subject has one or more risk factor(s) associated with acute myeloid leukemia. In some embodiments, a subject has two or more risk factor(s) associated with acute myeloid leukemia. In some embodiments, a subject has three or more risk factor(s) associated with acute myeloid leukemia. In some embodiments, a subject has four or more risk factor(s) associated with acute myeloid leukemia. In some embodiments, a subject has five or more risk factor(s) associated with acute myeloid leukemia. In some embodiments, a subject has six or more risk factor(s) associated with acute myeloid leukemia. In some embodiments, a subject has seven or more risk factor(s) associated with acute myeloid leukemia. In some embodiments, a subject has eight or more risk factor(s) associated with acute myeloid leukemia. In some embodiments, a subject has nine or more risk factor(s) associated with acute myeloid leukemia. In some embodiments, a subject has ten or more risk factor(s) associated with acute myeloid leukemia.

The present disclosure, in some aspects, provides methods of inhibiting the progression from clonal hematopoiesis to myeloid malignancy. The progression from clonal hematopoiesis to myeloid malignancy may occur because of clonal expansion progressing toward hematological abnormality or leukemic transformation. Myeloid malignancies are a heterogeneous group of clonal disorders, characterized by excessive proliferation, abnormal self-renewal, and/or differentiation defects of hematopoietic cells and myeloid progenitor cells. They include myeloproliferative neoplasms (MPNs), myelodysplastic syndromes (MDS), and AML. Briefly, sources of mutagenic stress (e.g., chemotherapy, aging or exogenous stress) may generate somatic variation in clonal hematopoiesis. By selective pressure, the somatic variants may be selected for if they have better fitness compared to non-variants. Selective pressures may lead to clonal outgrowth and the development of clonal hematopoiesis. In some instances, somatic variants can also lead directly to de novo AML formation. clonal hematopoiesis may be followed by the development of cytopenias. In some instances, AML development because of clonal hematopoiesis can arise from an myelodysplastic syndrome (MDS) intermediate but may also bypass this progression.

### III. Cellular Senescence

Aspects of the present disclosure relate to inhibiting senescence of bone marrow stromal cells. As described herein, “senescence” or “cellular senescence” (used interchangeably herein), refers to a cell fate that has distinctive traits, for example, irreversible replicative arrest, sustained viability with resistance to apoptosis, and frequently, increased metabolic activity. Cell signaling pathways that contribute to a cell entering a senescent state may include signals related to tissue or cellular damage. For example, these signaling pathways may include, but are not limited to, DNA damage, telomeric uncapping or dysfunction, exposure to extracellular DNA, oncogene activation, replicative stress or inducers of proliferation (such as growth hormone/IGF-1), protein aggregates, misfolded proteins, failed protein removal through decreased autophagy, presence of advanced glycation end products (AGEs) due to the reaction of reducing sugars with amino groups in proteins (e.g. Hemoglobin A1c is an AGE), saturated lipids and other bioactive lipids (bradykines, certain prostaglandins, etc.), reactive metabolites (e.g. ROS, hypoxia or hyperoxia), mechanical stress (e.g. bone-on-bone stress in osteoarthritis or shear stress such as occurs on the venous side of AV fistulae for haemodialysis or around atherosclerotic plaques), inflammatory cytokines (e.g. TNFα), damage-associated molecular patterns (DAMPs, e.g. released intracellular contents signaling breakage of neighboring cells), and pathogen-associated molecular patterns (PAMPs, e.g. bacterial endotoxins). Thus, inhibiting (e.g., preventing or delaying) senescence of bone marrow stromal cells may be achieved by using a senolytic agent to prevent, for example, irreversible replicative arrest, sustained viability with resistance to apoptosis, and/or increased metabolic activity.

The present disclosure, in some aspects, provides methods of assaying a sample for the presence of a somatic mutation. Types of mutations that may be assayed are single base substitutions, insertions and deletions, duplications and translocations. Methods of detecting mutations may involve isolating DNA from a sample and subjecting the DNA to PCR. After PCR a number of assays may be performed to detect the presence of a mutation including, but not limited to denaturing gradient gel electrophoresis (DGGE), constant denaturing gel electrophoresis (CDGE), temporal temperature gradient gel electrophoresis (TTGE), single-strand conformation polymorphism (SSCP), protein truncation test (PTT), and high-resolution melt (HRM) analysis. In some embodiments, DGGE may be performed to detect the presence of a mutation. In some embodiments, CDGE may be performed to detect the presence of a mutation. In some embodiments, TTGE may be performed to detect the presence of a mutation. In some embodiments, SSCP may be performed to detect the presence of a mutation. In some embodiments, PTT may be performed to detect the presence of a mutation. In some embodiments, HRM may be performed to detect the presence of a mutation.

The present disclosure, in some aspects, provides methods of assaying a sample for increased expression of senescence markers. In some embodiments, assaying involves detecting the expression of at least one (e.g., 1, 2, 3, 4, or more) senescence markers in a sample and comparing it to a control. In some embodiments, assaying involves detecting the expression of at least 1 (e.g., at least 1, at least 2, at least 3, at least 4, at least 5, at least 6, at least 7, at least 8, at least 9, or at least 10) senescence markers in a sample. In some embodiments, assaying involves detecting the expression of at least 1, at least 2, at least 3, at least 4, at least 5, at least 6, at least 7, at least 8, at least 9, or at least 10 senescence markers in a sample. In some embodiments, assaying involves detecting the expression of at least 1 senescence marker in a sample. In some embodiments, assaying involves detecting the expression of at least 2 senescence markers in a sample. In some embodiments, assaying involves detecting the expression of at least 3 senescence markers in a sample. In some embodiments, assaying involves detecting the expression of at least 4 senescence markers in a sample. In some embodiments, assaying involves detecting the expression of at least 5 senescence markers in a sample. In some embodiments, assaying involves detecting the expression of at least 6 senescence markers in a sample. In some embodiments, assaying involves detecting the expression of at least 7 senescence markers in a sample. In some embodiments, assaying involves detecting the expression of at least 8 senescence markers in a sample. In some embodiments, assaying involves detecting the expression of at least 9 senescence markers in a sample. In some embodiments, assaying involves detecting the expression of at least 10 senescence markers in a sample.

In some embodiments, the detection may include staining for a senescence marker using a fluorescent marker and visualizing the results using a plate reader or measured by flow cytometry or epifluorescence microscopy. Senescence markers may be any senescence associated secretory phenotype (SASP) markers. Senescence markers may be selected from but are not limited to senescence-associated β-galactosidase (SA-β-Gal), cyclin-dependent kinase inhibitor 2a (Cdkn2a) (P16), Cdknla (P21), Cdkn1b (P27), interleukin-6 (IL-6), IL-1α, IL-7, IL-1β, IL-13, IL-15, IL-8, growth-regulated oncogene-alpha (GRO-α), GRO-β, GRO-g, Monocyte chemotactic protein-2 (MCP-2), MCP-4, MIP-1a, MIP-3a, HCC-4, Eotaxin, Eotaxin-3, TECK, ENA-78, I-309, I-TAC, GM-CSE, G-CSE, IFN-γ, BLC, MIF, Amphiregulin, Epiregulin, Heregulin, EGF, bFGF, HGF, KGF (FGF7), VEGF, Angiogenin, SCF, SDF-1, PIGF, NGF, insulin-like growth factor binding protein 2 (IGFBP-2), IGFBP-3, IGFBP-4, IGFBP-6, IGFBP-7, matrix metalloproteinase-1 (MMP-1), MMP-3, MMP-10, MMP-12, MMP-13, MMP-14, tissue inhibitor of metalloproteinase 1 (TIMP-1), TIMP-2, PAI-1, PAI-2; tPA; uPA, Cathepsin B, ICAM-1, ICAM-3, OPG, STNFRI, TRAIL-R3, Fas, STNFRII, Fas, uPAR, SGP130, EGF-R, PGE2, Nitric oxide, Reactive oxygen species, Fibronectin, Collagens, and Laminin (see, e.g., Xu J et al. Front Pharmacol. 2020; 11: 601325). In some embodiments, the method comprises assaying a sample from the subject increased expression of SA-β-Gal, relative to a control. In some embodiments, the method comprises assaying a sample from the subject increased expression of P16, relative to a control. In some embodiments, the method comprises assaying a sample from the subject increased expression of P21, relative to a control. In some embodiments, the method comprises assaying a sample from the subject increased expression of P27, relative to a control. In some embodiments, the method comprises assaying a sample from the subject increased expression of IL-6, relative to a control. In some embodiments, the method comprises assaying a sample from the subject increased expression of IL-1a, relative to a control. In some embodiments, the method comprises assaying a sample from the subject increased expression of IL-7, relative to a control. In some embodiments, the method comprises assaying a sample from the subject increased expression of IL-1B, relative to a control. In some embodiments, the method comprises assaying a sample from the subject increased expression of IL-13, relative to a control. In some embodiments, the method comprises assaying a sample from the subject increased expression of IL-15, relative to a control. In some embodiments, the method comprises assaying a sample from the subject increased expression of IL-8, relative to a control. In some embodiments, the method comprises assaying a sample from the subject increased expression of GRO-α, relative to a control. In some embodiments, the method comprises assaying a sample from the subject increased expression of GRO-β, relative to a control. In some embodiments, the method comprises assaying a sample from the subject increased expression of GRO-g, relative to a control. In some embodiments, the method comprises assaying a sample from the subject increased expression of MCP-2, relative to a control. In some embodiments, the method comprises assaying a sample from the subject increased expression of MCP-4, relative to a control. In some embodiments, the method comprises assaying a sample from the subject increased expression of MIP-1α, relative to a control. In some embodiments, the method comprises assaying a sample from the subject increased expression of MIP-3a, relative to a control. In some embodiments, the method comprises assaying a sample from the subject increased expression of HCC-4, relative to a control. In some embodiments, the method comprises assaying a sample from the subject increased expression of Eotaxin, relative to a control. In some embodiments, the method comprises assaying a sample from the subject increased expression of Eotaxin-3, relative to a control. In some embodiments, the method comprises assaying a sample from the subject increased expression of TECK, relative to a control. In some embodiments, the method comprises assaying a sample from the subject increased expression of ENA-78, relative to a control. In some embodiments, the method comprises assaying a sample from the subject increased expression of I-309, relative to a control. In some embodiments, the method comprises assaying a sample from the subject increased expression of I-TAC, relative to a control. In some embodiments, the method comprises assaying a sample from the subject increased expression of GM-CSE, relative to a control. In some embodiments, the method comprises assaying a sample from the subject increased expression of G-CSE, relative to a control. In some embodiments, the method comprises assaying a sample from the subject increased expression of IFN-γ, relative to a control. In some embodiments, the method comprises assaying a sample from the subject increased expression of BLC, relative to a control. In some embodiments, the method comprises assaying a sample from the subject increased expression of MIF, relative to a control. In some embodiments, the method comprises assaying a sample from the subject increased expression of Amphiregulin, relative to a control. In some embodiments, the method comprises assaying a sample from the subject increased expression of Epiregulin, relative to a control. In some embodiments, the method comprises assaying a sample from the subject increased expression of Heregulin, relative to a control. In some embodiments, the method comprises assaying a sample from the subject increased expression of EGF, relative to a control. In some embodiments, the method comprises assaying a sample from the subject increased expression of bFGF, relative to a control. In some embodiments, the method comprises assaying a sample from the subject increased expression of HGF, relative to a control. In some embodiments, the method comprises assaying a sample from the subject increased expression of KGF (FGF7), relative to a control. In some embodiments, the method comprises assaying a sample from the subject increased expression of VEGF, relative to a control. In some embodiments, the method comprises assaying a sample from the subject increased expression of Angiogenin, relative to a control. In some embodiments, the method comprises assaying a sample from the subject increased expression of SCF, relative to a control. In some embodiments, the method comprises assaying a sample from the subject increased expression of SDF-1, relative to a control. In some embodiments, the method comprises assaying a sample from the subject increased expression of PIGF, relative to a control. In some embodiments, the method comprises assaying a sample from the subject increased expression of NGF, relative to a control. In some embodiments, the method comprises assaying a sample from the subject increased expression of IGFBP-2, relative to a control. In some embodiments, the method comprises assaying a sample from the subject increased expression of IGFBP-3, relative to a control. In some embodiments, the method comprises assaying a sample from the subject increased expression of IGFBP-4, relative to a control. In some embodiments, the method comprises assaying a sample from the subject increased expression of IGFBP-6, relative to a control. In some embodiments, the method comprises assaying a sample from the subject increased expression of IGFBP-7, relative to a control. In some embodiments, the method comprises assaying a sample from the subject increased expression of MMP-1, relative to a control. In some embodiments, the method comprises assaying a sample from the subject increased expression of MMP-3, relative to a control. In some embodiments, the method comprises assaying a sample from the subject increased expression of MMP-10, relative to a control. In some embodiments, the method comprises assaying a sample from the subject increased expression of MMP-12, relative to a control. In some embodiments, the method comprises assaying a sample from the subject increased expression of MMP-13, relative to a control. In some embodiments, the method comprises assaying a sample from the subject increased expression of MMP-14, relative to a control. In some embodiments, the method comprises assaying a sample from the subject increased expression of TIMP-1, relative to a control. In some embodiments, the method comprises assaying a sample from the subject increased expression of TIMP-2, relative to a control. In some embodiments, the method comprises assaying a sample from the subject increased expression of PAI-1, relative to a control. In some embodiments, the method comprises assaying a sample from the subject increased expression of PAI-2, relative to a control. In some embodiments, the method comprises assaying a sample from the subject increased expression of tPA, relative to a control. In some embodiments, the method comprises assaying a sample from the subject increased expression of uPA, relative to a control. In some embodiments, the method comprises assaying a sample from the subject increased expression of Cathepsin B, relative to a control. In some embodiments, the method comprises assaying a sample from the subject increased expression of ICAM-1, relative to a control. In some embodiments, the method comprises assaying a sample from the subject increased expression of ICAM-3, relative to a control. In some embodiments, the method comprises assaying a sample from the subject increased expression of OPG, relative to a control. In some embodiments, the method comprises assaying a sample from the subject increased expression of sTNFRI, relative to a control. In some embodiments, the method comprises assaying a sample from the subject increased expression of TRAIL-R3, relative to a control. In some embodiments, the method comprises assaying a sample from the subject increased expression of Fas, relative to a control. In some embodiments, the method comprises assaying a sample from the subject increased expression of sTNFRII, relative to a control. In some embodiments, the method comprises assaying a sample from the subject increased expression of Fas, relative to a control. In some embodiments, the method comprises assaying a sample from the subject increased expression of uPAR, relative to a control. In some embodiments, the method comprises assaying a sample from the subject increased expression of SGP130, relative to a control. In some embodiments, the method comprises assaying a sample from the subject increased expression of EGF-R, relative to a control. In some embodiments, the method comprises assaying a sample from the subject increased expression of PGE2, relative to a control. In some embodiments, the method comprises assaying a sample from the subject increased expression of Nitric oxide, relative to a control. In some embodiments, the method comprises assaying a sample from the subject increased expression of Reactive oxygen species, relative to a control. In some embodiments, the method comprises assaying a sample from the subject increased expression of Fibronectin, relative to a control. In some embodiments, the method comprises assaying a sample from the subject increased expression of Collagens, relative to a control. In some embodiments, the method comprises assaying a sample from the subject increased expression of Laminin, relative to a control. In some embodiments, the method comprises assaying a sample from the subject increased expression of senescence-associated β-galactosidase (SA-β-Gal), Cdkn2a (P16), Cdkn1a (P21), Cdkn1b (P27), IL-6 and IL-1α, relative to a control.

The present disclosure, in some aspects, provides methods relating to the use of senolytic agents to inhibit senescence of bone marrow stromal cells. A senolytic agent is an agent that selectively kills senescent cells. In some embodiments, the senolytic agent is a class of drugs, small molecules or compounds that selectively kill senescent cells. A senolytic agent may also be in the form of a vaccine or a method of gene editing, for example, using CRISPR/Cas to modify specific genes to trigger apoptosis. In some embodiments, senolytic agents may delay, prevent, alleviate, or reverse age-related diseases. In some embodiments, senolytic agents may delay, prevent, alleviate, and reverse age-related diseases. In some embodiments, senolytic agents may delay, prevent, alleviate, and/or reverse age-related diseases.

In some embodiments, the senolytic agent is a class of drugs that selectively kill senescent cells. In some embodiments, the senolytic agent is a class of small molecules that selectively kill senescent cells. In some embodiments, the senolytic agent is a class of compounds that selectively kill senescent cells. In some embodiments, the senolytic agent is a class of drugs, small molecules or compounds that selectively kill senescent cells. In some embodiments, the senolytic agent is a class of drugs, small molecules and compounds that selectively kill senescent cells. In some embodiments, the senolytic agent is a class of drugs, small molecules and/or compounds that selectively kill senescent cells. In some embodiments, the senolytic agent is a vaccine or a method of gene editing (e.g., CRISPR/Cas). In some embodiments, the senolytic agent is a vaccine and a method of gene editing (e.g., CRISPR/Cas). In some embodiments, the senolytic agent is a vaccine and/or a method of gene editing (e.g., CRISPR/Cas). In some embodiments, the senolytic agent is a vaccine. In some embodiments, the senolytic agent is a method of gene editing (e.g., CRISPR/Cas).

Senolytic agents may be selected from, but are not limited to, dasatinib, quercetin, fisetin, piperlongumine, azithromycin, roxithromycin, navitoclax (ABT-263), luteolin, curcumin, curcumin analog EF24, A1331852, A1155463, geldanamycin, tanespimycin, nutlin3a, FOXO4-related peptides, BCL-2 inhibitors, Src inhibitors, USP7 inhibitors, SSK1, BIRC5 knockout, GLS1 inhibitors, Anti-GPNMB vaccine, cardiac glycosides (e.g., ouabain, proscillaridin a, digoxin), 25-hydroxycholesterol (25HC), procyanidin C1, 17-DMAG (Alvespimycin), catechins (e.g., SUNPHENON®) and EF-24.

In some embodiments, the senolytic agent is dasatinib (SPRYCEL®) (CAS No. 302962-49-8)), as shown in Formula I:

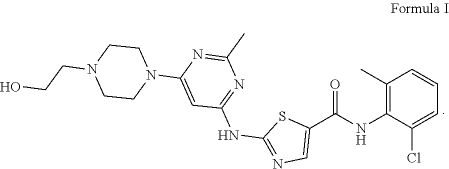 [SMILES: Cc1nc(Nc2ncc(C(=O)Nc3c(C)cccc3Cl)s2)cc(N2CCN(CCO)CC2)n1]

In some embodiments, dasatinib is administered to a subject at a dose of about 100 mg to about 150 mg, daily, weekly or monthly.

In some embodiments, the senolytic agent is quercetin (CAS No. 117-39-5), as shown in Formula II:

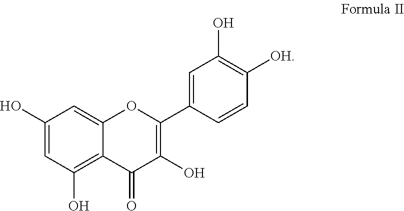 [SMILES: O=c1c(O)c(-c2ccc(O)c(O)c2)oc2cc(O)cc(O)c12]

In some embodiments, quercetin is administered to a subject at a dose of about 100 mg to about 500 mg, daily, weekly or monthly.

In some embodiments, the senolytic agent is 17-DMAG (Alvespimycin) (CAS No. 467214-21-7) as shown in Formula III:

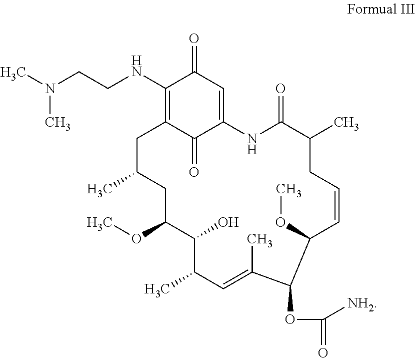 [SMILES: CO[C@H]1/C=C\CC(C)C(=O)NC2=CC(=O)C(NCCN(C)C)=C(C[C@@H](C)C[C@H](OC)[C@H](O)[C@@H](C)/C=C(\C)[C@@H]1OC(N)=O)C2=O]

In some embodiments, Alvespimycin is administered to a subject at a dose of about 50 mg to about 1000 mg (e.g., about 500 mg to a bout 100-mg), daily, weekly or monthly.

In some embodiments, the senolytic agent is fisetin (CAS No. 528-48-3) as shown in Formula IV:

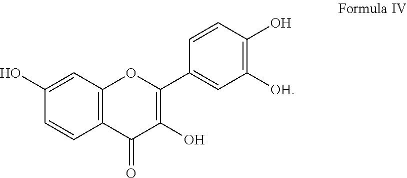 [SMILES: O=c1c(O)c(-c2ccc(O)c(O)c2)oc2cc(O)ccc12]

In some embodiments, fisetin is administered to a subject at a dose of about 100 mg to about 1000 mg (e.g., about 500 mg to a bout 100-mg), daily, weekly or monthly.

In some embodiments, the senolytic agent is catechins (SUNPHENON®) (CAS No. 7295-85-4) as shown in Formula V:

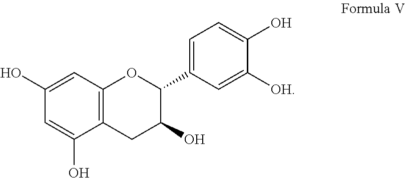 [SMILES: Oc1cc(O)c2c(c1)O[C@H](c1ccc(O)c(O)c1)[C@@H](O)C2]

In some embodiments, catechins (SUNPHENON®) is administered to a subject at a dose of about 50 mg to about 500 mg (e.g., about 500 mg to a bout 100-mg), daily, weekly or monthly.

In some embodiments, the senolytic agent is navitoclax (CAS No. 923564-51-6) as shown in Formula VI:

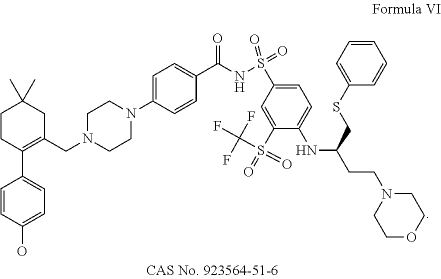 [SMILES: CC1(C)CCC(c2ccc(O)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, navitoclax is administered to a subject at a dose of about 50 mg to about 1000 mg (e.g., about 500 mg to a bout 100-mg), daily, weekly or monthly.

### III. Methods of Treatment

A “subject in need thereof” refers to a subject in need of treatment for clonal hematopoiesis (e.g., AML, senescence of bone marrow stromal cells). In some embodiments, the subject in need may be one that is “non-responsive” or “refractory” to a standard therapy for the clonal hematopoiesis (e.g., AML, senescence of bone marrow stromal cells). In some embodiments, the terms “non-responsive” and “refractory” refers to the subject's response to therapy as not clinically adequate to relieve one or more symptoms associated with the neurological disease or disorder.

A subject generally refers to a mammal. The mammal may be, for example, a human, primate, mouse, rat, dog, cat, cow, horse, goat, camel, sheep, or a pig. In some embodiments, the subject is a human. In some embodiments, the subject is a primate. In some embodiments, the subject is a primate. In some embodiments, the subject is a mouse. In some embodiments, the subject is a rat. In some embodiments, the subject is a dog. In some embodiments, the subject is a cat. In some embodiments, the subject is a cow. In some embodiments, the subject is a horse. In some embodiments, the subject is a goat. In some embodiments, the subject is a camel. In some embodiments, the subject is a sheep. In some embodiments, the subject is a pig.

In some embodiments, a subject has AML. In some embodiments, the subject is at risk of blood cancer (e.g., AML). In some embodiments, the subject exhibits one or more symptoms of AML. In some embodiments, the subject exhibits one or more risk factors for AML.

In some embodiments, the subject is about 50 (e.g., about 48, about 49, about 50, about 51, about 52, about 55, about 60, about 65, about 70, about 75, about 80, about 85, about 90, about 95) years old or older. In some embodiments, the subject is about 48, about 49, about 50, about 51, about 52, about 55, about 60, about 65, about 70, about 75, about 80, about 85, about 90, about 95 years of older. In some embodiments, the subject is about 48 years old. In some embodiments, the subject is about 49 years old. In some embodiments, the subject is about 50 years old. In some embodiments, the subject is about 51 years old. In some embodiments, the subject is about 52 years old. In some embodiments, the subject is about 55 years old. In some embodiments, the subject is about 60 years old. In some embodiments, the subject is about 65 years old. In some embodiments, the subject is about 70 years old. In some embodiments, the subject is about 75 years old. In some embodiments, the subject is about 80 years old. In some embodiments, the subject is about 85 years old. In some embodiments, the subject is about 90 years old. In some embodiments, the subject is about 70 years old. In some embodiments, the subject is about 95 years old.

In some embodiments, a subject at risk of blood cancer (e.g., AML) exhibits a sign of clonal hematopoiesis. This includes subjects with age-related mutations associated with clonal hematopoietic expansion and malignancies, mainly DNMT3A, TET2, and ASXL1. In some embodiments, the subject has a mutation in DNMT3A. In some embodiments, the subject has a mutation in TET2. In some embodiments, the subject has a mutation in ASXL1.

In some embodiments, inhibition of clonal hematopoiesis may be measured relative to a control. A “control” may be a subject that does not have a somatic mutation (e.g., DNMT3A) that leads to aberrant clonal hematopoiesis. A control may be a subject that exhibits low levels of clonal hematopoiesis. A control may be a subject that does not exhibit symptoms of myeloid malignancy. A control may be a subject that exhibits little to no expression of senescent markers, or expression above or below a threshold value.

“An effective amount” is an amount that alleviates one or more symptom(s) associated with a particular condition, such as clonal hematopoiesis and/or AML (or other blood cancer). In some embodiments, an effective amount inhibits myeloproliferation of bone marrow stromal cells. In some embodiments, an effective amount inhibits progression from clonal hematopoiesis to myeloid malignancy. “Inhibition” refers to at least 20% (e.g., at least 30%, at least 40%, at least 50%, at least 60%, at least 70%, at least 80%, or at least 90%) inhibition relative to a control. In some embodiments, inhibition is at least 20% (e.g., at least 20%, at least 30%, at least 40%, at least 50%, at least 60%, at least 70%, at least 80%, or at least 90%) inhibition relative to a control. In some embodiments, inhibition is at least 20%, at least 30%, at least 40%, at least 50%, at least 60%, at least 70%, at least 80%, or at least 90% inhibition relative to a control. In some embodiments, inhibition is at least 20% inhibition relative to a control. In some embodiments, inhibition is at least 30% inhibition relative to a control. In some embodiments, inhibition is at least 40% inhibition relative to a control. In some embodiments, inhibition is at least 50% inhibition relative to a control. In some embodiments, inhibition is at least 60% inhibition relative to a control. In some embodiments, inhibition is at least 70% inhibition relative to a control. In some embodiments, inhibition is at least 80% inhibition relative to a control. In some embodiments, inhibition is at least 90% inhibition relative to a control.

In some embodiments, the senolytic agent is formulated in one or more composition(s) (e.g., different or same compositions) for administration to a subject. The composition(s) may take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intraosseous, intrapleural, intrathecal, transdermal, transmucosal, rectal, and the like). In some embodiments, the composition(s) are in the form of liquids, aerosols, solutions, inhalants, mists, sprays, or solids, powders, ointments, pastes, creams, lotions, gels, or patches. In some embodiments, the composition(s) are in the form of liquids, aerosols, solutions, inhalants, mists, sprays, or solids, powders, ointments, pastes, creams, lotions, gels, and/or patches. In some embodiments, the composition(s) are in the form of liquids. In some embodiments, the composition(s) are in the form of aerosols. In some embodiments, the composition(s) are in the form of solutions. In some embodiments, the composition(s) are in the form of inhalants. In some embodiments, the composition(s) are in the form of mists. In some embodiments, the composition(s) are in the form of sprays. In some embodiments, the composition(s) are in the form of solids. In some embodiments, the composition(s) are in the form of powders. In some embodiments, the composition(s) are in the form of ointments. In some embodiments, the composition(s) are in the form of pastes. In some embodiments, the composition(s) are in the form of creams. In some embodiments, the composition(s) are in the form of lotions. In some embodiments, the composition(s) are in the form of gels. In some embodiments, the composition(s) are in the form of patches.

In some embodiments, a pharmaceutical composition of the disclosure may be in the form of an aqueous solution or powder for aerosol administration by inhalation or insufflation (cither through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration. In some embodiments, a pharmaceutical composition of the disclosure may be in the form of an aqueous solution and/or powder for aerosol administration by inhalation and/or insufflation. In some embodiments, a pharmaceutical composition of the disclosure may be in the form of an aqueous solution for aerosol administration by inhalation. In some embodiments, a pharmaceutical composition of the disclosure may be in the form of an aqueous solution for aerosol administration by insufflation. In some embodiments, a pharmaceutical composition of the disclosure may be in the form of a powder solution for aerosol administration by inhalation. In some embodiments, a pharmaceutical composition of the disclosure may be in the form of a powder solution for aerosol administration by insufflation. In some embodiments, a pharmaceutical composition of the disclosure may be in the form of a tablet and/or capsule for oral administration. In some embodiments, a pharmaceutical composition of the disclosure may be in the form of a tablet for oral administration. In some embodiments, a pharmaceutical composition of the disclosure may be in the form of a capsule for oral administration. In some embodiments, a pharmaceutical composition of the disclosure may be in the form of a sterile aqueous solution and/or dispersion suitable for administration by either direct injection or by addition to sterile infusion fluids for intravenous infusion. In some embodiments, a pharmaceutical composition of the disclosure may be in the form of a sterile aqueous solution for administration by direct injection. In some embodiments, a pharmaceutical composition of the disclosure may be in the form of a sterile aqueous solution for administration by addition to sterile infusion fluids for intravenous infusion. In some embodiments, a pharmaceutical composition of the disclosure may be in the form of a dispersion suitable for administration by direct injection. In some embodiments, a pharmaceutical composition of the disclosure may be in the form of a dispersion suitable for administration by addition to sterile infusion fluids for intravenous infusion. In some embodiments, a pharmaceutical composition of the disclosure may be in the form of a lotion for transdermal administration. In some embodiments, a pharmaceutical composition of the disclosure may be in the form of a cream for transdermal administration. In some embodiments, a pharmaceutical composition of the disclosure may be in the form of a foam for transdermal administration. In some embodiments, a pharmaceutical composition of the disclosure may be in the form of a patch for transdermal administration. In some embodiments, a pharmaceutical composition of the disclosure may be in the form of a suspension for transdermal administration. In some embodiments, a pharmaceutical composition of the disclosure may be in the form of a solution for transdermal administration. In some embodiments, a pharmaceutical composition of the disclosure may be in the form of a suppository for transdermal administration. In some embodiments, a pharmaceutical composition of the disclosure may be in the form of a lotion for transmucosal administration. In some embodiments, a pharmaceutical composition of the disclosure may be in the form of a cream for transmucosal administration. In some embodiments, a pharmaceutical composition of the disclosure may be in the form of a foam for transmucosal administration. In some embodiments, a pharmaceutical composition of the disclosure may be in the form of a patch for transmucosal administration. In some embodiments, a pharmaceutical composition of the disclosure may be in the form of a suspension for transmucosal administration. In some embodiments, a pharmaceutical composition of the disclosure may be in the form of a solution for transmucosal administration. In some embodiments, a pharmaceutical composition of the disclosure may be in the form of a suppository for transmucosal administration.

In some embodiments, the administration route of the senolytic agents may be intravenous. In some embodiments, the administration route of the senolytic agents may be intraosseous. In some embodiments, the senolytic agents may be administered in either continuous or intermittently. In some embodiments, the senolytic agents may be administered continuously. In some embodiments, the senolytic agents may be administered intermittently.

### EXAMPLES

### Example 1. Bone Marrow Stromal Cells (BMSC) Gain an Enriched Gene Signature of Cellular Senescence in the Context of Dumt3a Mutant Hematopoiesis

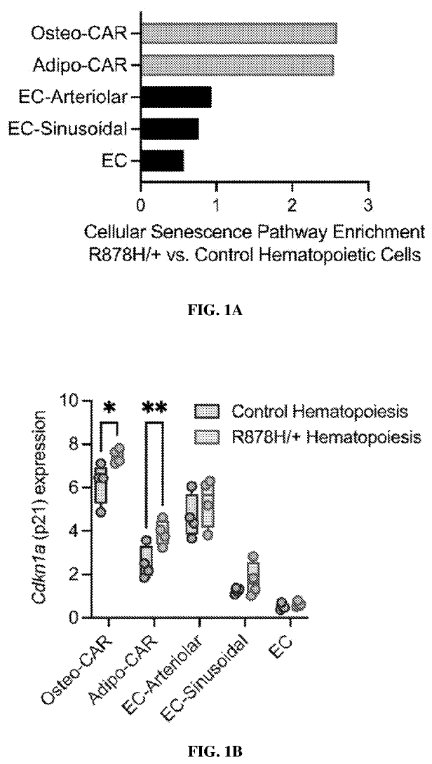

Clonal haematopoiesis (clonal hematopoiesis) is an age-related condition caused by somatic mutation(s) that give a haematopoietic stem cell (HSC) a clonal selective advantage. The most frequently mutated gene in human clonal hematopoiesis and acute myeloid leukaemia (AML) is DNA methyltransferase 3A (DNMT3A). The precise mechanisms by which the DNMT3A mutation confers a clonal advantage and leads to development of AML is unknown. We performed single-cell RNA-sequencing (RNA-seq) of the hematopoietic and non-hematopoietic bone marrow (BM) fractions of a murine model of the DNMT3A hotspot mutation. Surprisingly, we found that subsets of bone marrow stromal cells (BMSC) gain an enriched gene signature of cellular senescence and have increased P21 expression in the context of Dnmt3a-mutant haematopoiesis (FIG. 1A-1B).

First, we investigated the effects of Dnmt3a-mutant hematopoietic cells on wild type non-hematopoietic cells of the BM microenvironment. We transplanted Dnmt3a-mutant (R878H/+) BM cells or control (wild-type Dnmt3a) BM cells into mid-aged wild type recipient mice and performed single cell RNA-seq on enriched hematopoietic (CD45+) and non-hematopoietic (CD45− Ter119−) BM cell fractions. Examining markers of senescence in the subsets of BMSCs that were profiled, we observed strong enrichment of a cellular senescence gene signature in Osteo-CAR (“Cxcl12-abundant reticular”) and Adipo-CAR cells following exposure to Dnmt3a-mutant hematopoietic cells (FIG. 1A). This gene expression program included increased expression of the canonical senescence-associated gene Cdkn1a (p21) in mutant Osteo-CAR and Adipo-CAR cells relative to controls (FIG. 1B). Additionally, endothelial cells (EC), EC-Arteriolar, and EC-Sinusoidal did not exhibit increased Cdkn1a (p21) expression. This led us to hypothesize that Dnmt3a-mutant HSCs remodel the BM microenvironment through induction of senescence.

### Example 2. Dnmt3a Mutant Hematopoietic Stem and Progenitor Cells (HSPC) Induce Senescence to Favor Clonal Expansion and Transformation to Acute Myeloid Leukemia

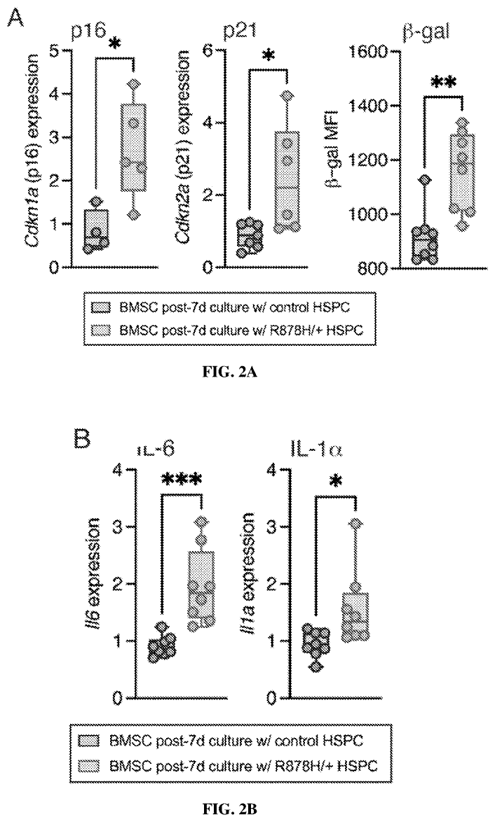

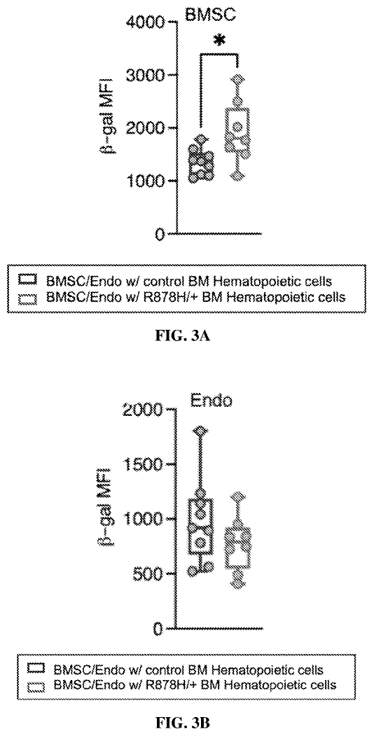

We tested whether Dnmt3a-mutant haematopoietic stem and progenitor cells (HSPC) induce senescence of the BM microenvironment to favour clonal expansion and transformation to AML. Ex vivo cocultures show BMSC cultured with Dnmt3a-mutant HSPC have increased expression of senescence markers (P16, P21, β-gal) and senescence associated secretory phenotype (IL-6, IL-1a) compared to BMSC cultured with control wild type (WT) HSPC (FIG. 2A-2B). A similar phenotype was observed with Dnmt3a-mutant cells transplanted into WT recipient mice; this resulted in increased β-gal staining in BMSCs, which was not observed in the endothelial cells (FIG. 3A-3B).

Using ex vivo co-culture of BMSC with either Dnmt3a-mutant (R878H/+) HSPC or control HSPC, we found that after 7 days (“post-7d”) Dnmt3a-mutant HSPCs caused increased expression of senescence markers Cdkn2a (p16), Cdkn1a (p21) and β-galactosidase (β-gal) in primary wild type BMSCs relative to BMSC that were cultured with control HSPCs (FIG. 2A). Increased expression of Il6 and Il1a, components of the senescence-associated secretory phenotype (SASP), were also observed in BMSC cultured with R878H/+ HSPC relative to BMSC cultured with control HSPC (FIG. 2B).

In vivo transplant experiments were performed using either control (wild-type Dnmt3a) BM hematopoietic cells or Dnmt3a-mutant (R878H/+) BM hematopoictic cells. The data showed increased intracellular β-gal in BMSCs with transplanted Dnmt3a-mutant hematopoietic cells relative to BMSCs with transplanted control BM hematopoietic cells (FIG. 3A) but not in the endothelial cells transplanted with either control BM hematopoietic cells or Dnmt3a-mutant hematopoictic cells (FIG. 3B) 12 weeks after transplant of Dnmt3a-mutant or control BM cells.

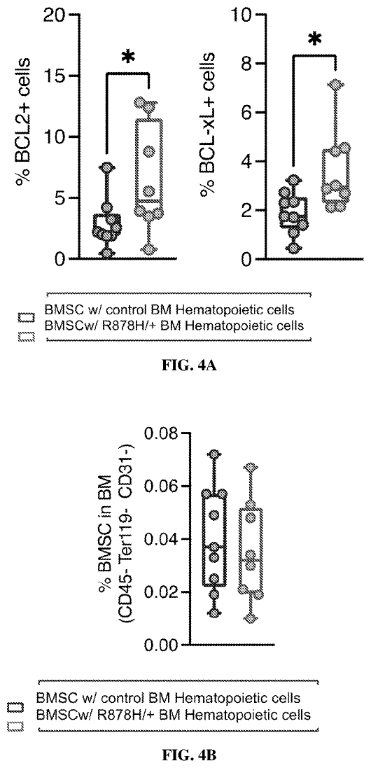

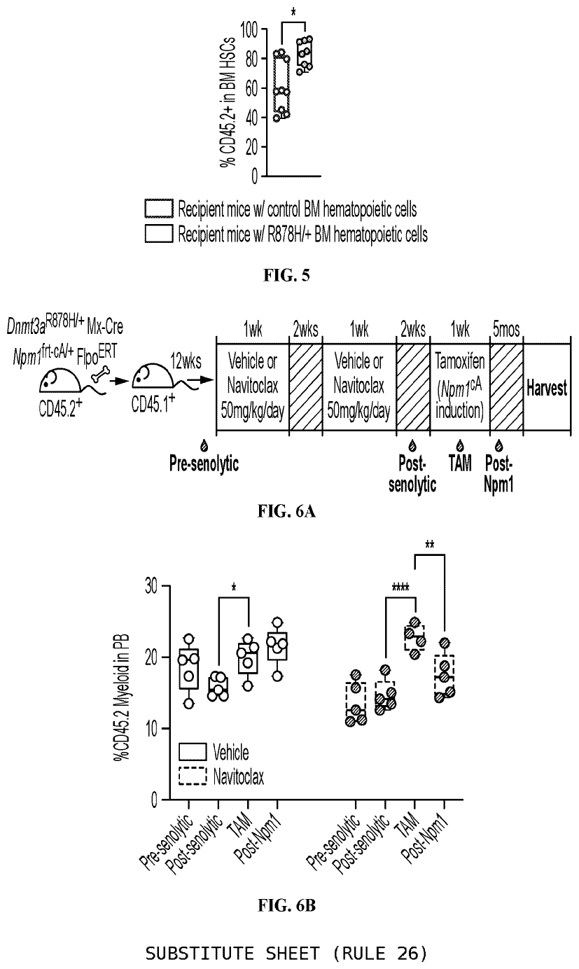

Moreover, BCL2 and BCLxL expression were found to be increased in the BMSCs, without altering the total numbers of BMSCs (FIG. 4A-4B). In these mice, Dnmt3a-mutant HSCs were expanded relative to control HSCs (FIG. 5).

In in vivo transplant experiments also showed increased intracellular expression of the senescence-associated anti-apoptotic proteins Bcl-2 and Bcl-xL in BMSCs 12 weeks after transplant of Dnmt3a-mutant cells relative to transplant of control BM cells (FIG. 4A) without altering the total number of BMSCs (FIG. 4B). Furthermore, in these animals, Dnmt3a-mutant HSCs were expanded compared to control HSCs (FIG. 5). This supports a positive correlation between BMSC senescence and the expansion of Dnmt3a-mutant HSC in vivo.

### Example 3. Dnmt3a Mutant Hematopoietic Stem and Progenitor Cells (HSPC) Induce Senescence to Favor Clonal Expansion and Transformation to Acute Myeloid Leukemia

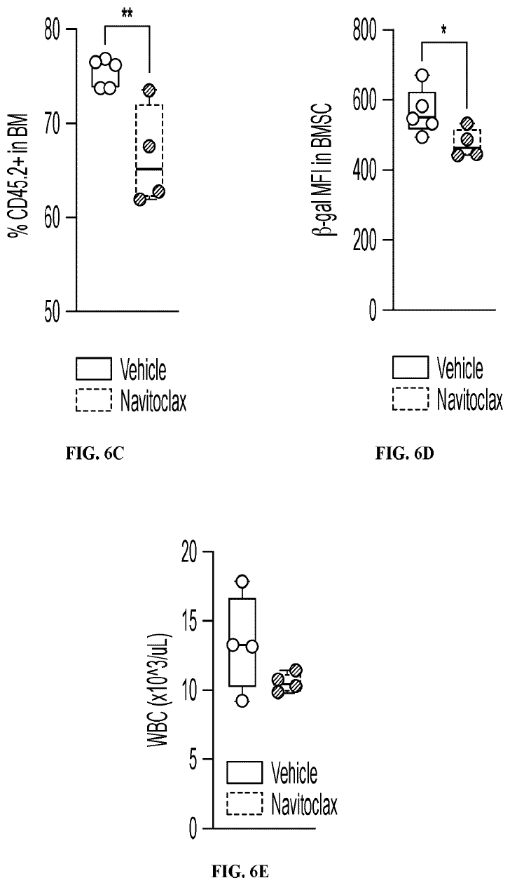

Next, the functional importance of BMSC senescence in Dnmt3a-mutant transformation to AML was evaluated. Dnmt3a-mutant cells were transplanted into WT recipient mice, administered the senolytic Navitoclax or vehicle control, and then induced the AML driver mutation Npm1^{c} in vivo. We observed Navitoclax treatment reduced peripheral blood myeloid cell overproduction concurrent with transformation of Dnmt3a; Npm1-mutant cells, suggesting senolytics may delay AML development (FIG. 6A-6E).

Using the senolytic Navitoclax, targeting Bcl-2/Bcl-xL, we evaluated the extent to which removing senescent cells caused by Dnmt3a-mutant haematopoiesis in vivo would delay progression from clonal hematopoiesis to myeloid malignancy. Briefly, Dnmt3a-mutant cells (cells from BM derived from CD45.2⁺; Dnmt3a^{R787H/+} MxCre; Npm1^{frt-cA/+}Flpo^{ERT} mice) were transplanted into recipient mice (CD45.1⁺ mice) (FIG. 6A). After 12 weeks (‘Pre-senolytic’), recipient mice were either administered vehicle control or the senolytic agent, navitoclax, by oral gavage to selectively deplete senescent cells. Navitoclax was administered at a dosage of 50 mg/kg/day for 1 week, followed by 2 weeks of no treatment, followed by 1 week of treatment. Blood samples were collected from Pre-senolytic mice before administration of vehicle control or navitoclax. Following navitoclax treatment (‘Post-senolytic’), the AML driver mutation Npm1^{cA/+} (‘TAM’) was induced using tamoxifen to initiate progression to myeloid malignancy. Blood samples were collected from Post-senolytic mice and TAM mice. Tamoxifen was administered for a period of 1 week. Blood samples were taken from tamoxifen treated mice (“Post-Npm1”). The data shows that in the peripheral blood (“PB”) tamoxifen administration (“TAM”) increases the frequency of myeloid cells (CD45.2⁺) in both vehicle and navitoclax treatment conditions relative to post-senolytic samples. However, at subsequent sampling (‘Post-Npm1’), navitoclax-treated mice had reduced frequency of myeloid cells (CD45.2⁺) in the peripheral blood (PB) (FIG. 6B). At 5 months after tamoxifen treatment (“harvest”), navitoclax-treated mice had lower frequency of donor (CD45.2⁺) cells in the BM (FIG. 6C), maintained lower BMSC senescence (measured by β-gal) (FIG. 6D), and had reduced monocyte (“white blood cell” or “WBC”) count (FIG. 6E) relative to vehicle treated controls. While recipient mice transplanted with CD45.2⁺; Dnmt3a^{R787H/+} MxCre; Npm1^{frt-cA/+}Flpo^{ERT} cells had not yet developed to full myeloid malignancy, these results support removal of senescent cells induced by Dumt3a-mutant haematopoiesis using senolytic agents delay myeloproliferation that occurs during transformation from clonal hematopoiesis to AML.

Together, we demonstrated that Dnmt3a-mutant hematopoietic cells can induce BMSC senescence, and we nominate targeting the senescent BM microenvironment as a strategy to reduce clonal hematopoiesis and prevent transformation to AML.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A method of inhibiting clonal hematopoiesis in a subject in need thereof, the method comprising administering a senolytic agent to subject in an effective amount to inhibit senescence of bone marrow stromal cells in the subject, thereby inhibiting clonal hematopoiesis in the subject, relative to a control.

2. The method of claim 1, wherein the subject exhibits one or more symptom of acute myeloid leukemia.

3. The method of claim 1 or 2, wherein the subject has one or more known risk factors associated with acute myeloid leukemia.

4. The method of any one of the preceding claims, wherein the subject has acute myeloid leukemia.

5. The method of any one of the preceding claims, wherein the subject is 50 years old or older.

6. The method of any one of the preceding claims, wherein the effective amount of the senolytic agent inhibits myeloproliferation of the bone marrow stromal cells.

7. The method of any one of the preceding claims, wherein the effective amount of the senolytic agent inhibits progression from clonal hematopoiesis to myeloid malignancy, relative to a control.

8. The method of any one of the preceding claims, wherein hematopoietic stem and progenitor cells (HSPCs) of the subject comprise a somatic mutation that gives the HSPCs a clonal selective advantage.

9. The method of claim 8, wherein the mutation is a DNA methyltransferase 3A (DNMT3A) mutation.

10. The method of claim 8 or 9, further comprising assaying a sample from the subject for presence of the somatic mutation.

11. The method of any one of the preceding claims, further comprising assaying a sample from the subject increased expression of senescence markers, relative to a control.

12. The method of claim 11, wherein the senescence markers are selected from senescence-associated β-galactosidase (SA-8-Gal), Cdkn2a (P16), Cdkn1a (P21), Cdkn1b (P27), IL-6 and IL-1a.

13. The method of any one of the preceding claims, wherein the bone marrow stromal cells comprise adipo-Cxc112-abundant reticular (CAR) cells and osteo-CAR cells.

14. The method of any one of the preceding claims, wherein the senolytic agent is selected from dasatinib, quercetin, fisetin, 17-DMAG, navitoclax, and catechins.

15. The method of any one of the preceding claims, wherein the senolytic agent is administered via an intravenous route or an intraosseous route.

16. A method of inhibiting clonal hematopoiesis in a subject in need thereof, the method comprising:
assaying a biological sample obtained from the subject for a DNA methyltransferase 3A (DNMT3A) mutation; and
administering a senolytic agent to subject in an effective amount to inhibit senescence of bone marrow stromal cells in the subject, thereby inhibiting clonal hematopoiesis in the subject, relative to a control.

17. A method of inhibiting clonal hematopoiesis in a subject in need thereof, the method comprising:
assaying a biological sample obtained from the subject for a biomarker of cell senescence; and
administering a senolytic agent to subject in an effective amount to inhibit senescence of bone marrow stromal cells in the subject, thereby inhibiting clonal hematopoiesis in the subject, relative to a control.

18. The method of claim 16 or 17, wherein the subject is at risk for developing a myeloid malignancy.

19. The method of any one of claims 16-18, wherein the biological sample comprises hematopoietic cells.

20. The method of any one of claims 16-19, wherein the assaying comprises performing single-cell RNA sequencing (RNA-seq) on the biological sample.

21. The method of any one of claims 16-20, wherein the senolytic agent is selected from dasatinib, quercetin, fisetin, 17-DMAG, navitoclax, and catechins.
